(19) United States
(12) Patent Application Publication
(10) Pub. No.: US 2006/0184003 A1
(43) Pub. Date: Aug. 17, 2006

(54) Intra-procedurally determining the position of an internal anatomical target location using an externally measurable parameter

(76) Inventors: Jonathan S. Lewin, Baltimore, MD (US); Daniel Elgort, Cleveland, OH (US); Frank Wacker, Berlin (DE); Frank Sauer, Princeton, NJ (US); Ali Khamene, Princeton, NJ (US); Jeffrey L. Duerk, Avon Lake, OH (US)

Correspondence Address: MCDONALD HOPKINS CO., LPA, 2100 BANK ONE CENTER, 600 SUPERIOR AVENUE, E., CLEVELAND, OH, 44114-2653, US

(21) Appl. No.: 11/050,155

(22) Filed: Feb. 3, 2005

(51) Int. Cl. A61B 5/05

(52) U.S. Cl. 600/414

(57) ABSTRACT

Systems, methodologies, media, and other embodiments associated with facilitating intra-procedurally determining the position of an internal anatomical target location using an externally measurable parameter are described. One exemplary method embodiment includes pre-procedurally correlating internal anatomy motion with external marker motion. The example method may also include providing computer graphics to an augmented reality system during a percutaneous procedure to facilitate image guiding an interventional device with respect to the internal anatomy.

### FEDERAL FUNDING NOTICE

Portions of the claimed subject matter were developed with federal funding supplied under NIH Grants R01 CA81431-02 and R33 CA88144-01. The U.S. Government may have certain rights in the invention.

### TECHNICAL FIELD

The systems, methods, computer-readable media and so on described herein relate generally to the magnetic resonance imaging (MRI) arts. They find particular application to correlating and characterizing the position and/or movements of a region inside the body with the position and/or movements of markers and/or data provided by other apparatus outside the body.

### BACKGROUND

Some interventional procedures (e.g., needle biopsies, angiography) seek to access affected tissue while causing minimal injury to healthy tissue. The procedure may need to be applied to carefully selected and circumscribed areas. Therefore, monitoring the three dimensional position, orientation, and so on of an interventional device can facilitate a positive result. In these procedures, special instruments may be delivered to a subcutaneous target region via a small opening in the skin. The target region is typically not directly visible to an interventionalist and thus procedures may be performed using image guidance. In these image guidance systems, knowing the position of the instrument (e.g., biopsy needle, catheter tip) inside the patient and with respect to the target region helps achieving accurate, meaningful procedures. Thus, methods like stereotactic MRI guided breast biopsies have been developed. See, for example, U.S. Pat. No. 5,706,812.

These conventional image guidance methods facilitate making minimally invasive percutaneous procedures even less invasive. But these conventional MRI guided systems have typically required the procedure to take place within an imager and/or with repetitive trips into and out of an imager. These constraints have increased procedure time while decreasing ease-of-use and patient comfort. Furthermore, conventional systems may have required a patient to hold their breath or to be medicated to reduce motion due to respiration.

Additional real time in-apparatus image guided medical procedures are known in the art. For example, U.S. Published Application 20040034297, filed Aug. 12, 2002 describes systems and methods for positioning a medical device during imaging. Similarly, U.S. Published Application 20040096091, filed Oct. 10, 2003 describes a method and apparatus for needle placement and guidance in percutaneous procedures using real time MRI imaging. Likewise, U.S. Published Application 20040199067, filed Jan. 12, 2004 describes detecting the position and orientation of an interventional device within an MRI apparatus. These and similar methods and procedures require real time MRI imaging to guide a device. Indeed, the '067 publication recites that although it might be possible to find the position of an interventional device (e.g., biopsy needle) before a procedure by localizing it independent of MR (magnetic resonance) imaging using cameras and light emitting reflectors, the publication then points out that a free field of view between the reference markers and the camera would be required and that the field of view is limited when the interventional device is inside a patient body and thus the system will not work. Therefore, the '067 publication falls back onto real time imaging to guide a device during a procedure.

### BRIEF DESCRIPTION OF THE DRAWINGS

The accompanying drawings, which are incorporated in and constitute a part of the specification, illustrate various example systems, methods, and so on, that illustrate various example embodiments of aspects of the invention. It will be appreciated that the illustrated element boundaries (e.g., boxes, groups of boxes, or other shapes) in the figures represent one example of the boundaries. One of ordinary skill in the art will appreciate that in some examples one element may be designed as multiple elements, that multiple elements may be designed as one element, that an element shown as an internal component of another element may be implemented as an external component and vice versa, and so on. Furthermore, elements may not be drawn to scale.

FIG. 1 illustrates an example system configured to facilitate intra-procedurally determining the position of an internal anatomical target location using an externally measurable parameter.

FIG. 2 illustrates an example computer-executable method associated with providing real time computer graphics for guiding a percutaneous procedure without employing real time intra-procedural (e.g., MR) imaging.

FIG. 3 illustrates another example computer-executable method associated with providing real time computer graphics for guiding a percutaneous procedure without employing real time intra-procedural (e.g., MR) imaging.

FIG. 4 illustrates an example MRI apparatus configured to facilitate intra-procedurally determining the position of an anatomical target location using an externally measurable parameter.

FIG. 5 illustrates an example computer in which example systems and methods illustrated herein can operate, the computer being operably connectable to an MRI apparatus.

FIG. 6 illustrates an example 3d plot of MR marker positions acquired during an example pre-procedural respiratory cycle analysis.

FIG. 7 illustrates an example motion tracking marker.

FIG. 8 illustrates a subject with which a set of motion tracking markers has been associated.

FIG. 9 illustrates an interventional device to which a motion tracking marker has been attached.

FIG. 10 illustrates an example augmented reality system.

FIG. 11 illustrates an example screenshot from an example augmented reality system.

### DETAILED DESCRIPTION

Example systems and methods described herein concern pre-procedurally correlating internal anatomy position and/or movements with external marker position, external marker movements, and/or other externally measurable parameters to facilitate image-guiding percutaneous procedures outside an MR imager without acquiring real time images (e.g., MR images) during the procedures. Example systems and methods illustrate that in some examples the position and movements of a region (e.g., suspected tumor) inside a body (e.g., human, porcine) can be correlated to the position and movements of markers outside the body (e.g., on skin surface) with enough accuracy and precision (e.g., 2 mm) to facilitate image guiding procedures outside an imaging apparatus without real time imagery. Thus, minimally invasive procedures like needle biopsies may be image guided without having the patient in an imager (e.g., MRI apparatus) during the procedure.

In one example, image guiding may be provided by an augmented reality (AR) system that depends on correlations between pre-procedural images (e.g., MR images) and real time optical images (e.g., visible spectrum, infra red (IR)) acquired during a procedure. The pre-procedural images facilitate inferring, for example, organ motion and/or position even though the organs may move during a procedure. The organs may move due to, for example, respiration, cardiac activity, diaphragmatic activity, and so on. The organs may also move due to non-repetitive actions. Pre-procedural data may also include data from other apparatus. For example, pre-procedural data concerning cardiac motion may be acquired using an electrocardiogram (ECG), pre-procedural data concerning skeletal muscle motion may be acquired using an electromyogram (EMG), and so on.

In one example, pre-procedural images may include information concerning fixedly coupled MR/optical markers associated with (e.g., positioned on, attached to) a patient. Patient specific relationships concerning information in the pre-procedural MR images and/or other pre-procedural data (e.g., ECG data) can be analyzed pre-procedurally to determine correlations between the externally measurable parameters (e.g., reference marker locations) and anatomy of interest (e.g., region to biopsy). The correlations may therefore facilitate predicting the location of an anatomical target (e.g., tumor) at intervention time without performing real time imaging (e.g., MR imaging) during the intervention.

In one example, an interventional device (e.g., biopsy needle) may be configured with a set of visual reference markers. The visual reference markers may be rigidly and fixedly attached to the interventional device to facilitate visually establishing the three dimensional position and orientation of the interventional device. The position and orientation of the interventional device in a coordinate system that includes the fixedly coupled MR/optical reference markers and the subject may be determined during a device calibration operation. The fixedly coupled MR/optical reference markers may be left in place during a procedure and may therefore be tracked optically (e.g., in the near IR spectrum) during the procedure to provide feedback concerning motion due to, for example, respiration, cardiac activity, non-repetitive activity and so on. Then, also during the procedure, patient specific data that correlates reference marker position and/or movements with internal anatomy position and/or movements may be employed to facilitate inferring the location of the region of interest based on tracking the reference markers.

A calibration step may be performed pre-procedurally to facilitate establishing a transformation between, for example, external MR markers and external optical markers. The optical markers may then be tracked intra-procedurally. Based on the observed optical marker position, the transformation established between optical and MR markers during the calibration step, and the correlations between the position of the optical markers and the internal anatomical target, example systems can determine which pre-procedural MR image to display during a procedure. Once an appropriate MR image is selected, an example system may still need to align the MR image with the current position of the patient. Data acquired and relationships established during the calibration step facilitate this intra-procedural, real time alignment. Once again, while external markers are described, it is to be appreciated that data from other apparatus (e.g., ECG, respiration state monitor) may be acquired intra-procedurally and employed to select an appropriate pre-procedural MR image to display.

Thus, a pre-procedural MR image analyzed in light of externally measurable parameters (e.g., optically determined external marker positions) may facilitate providing an interventionalist (e.g., surgeon) with a visual image and other information (e.g., computer graphics) during the procedure without requiring intra-procedural (e.g., MR) imaging. In one example, an interventionalist may be provided with a display that includes the actual skin surface, an MRI slice at interesting level (e.g., device tip, tumor level), a graphical target (e.g., expanding/contracting bulls eye), a target path, an actual device track, a desired device track, a projected device track, and so on. In one example, the display may include a live stereoscopic video view of the actual observable scene, combined with overlaid MR images and computer graphics presented on a head-mountable augmented reality display.

The following includes definitions of selected terms employed herein. The definitions include various examples and/or forms of components that fall within the scope of a term and that may be used for implementation. The examples are not intended to be limiting. Both singular and plural forms of terms may be within the definitions.

“Percutaneous” means passed, done, or effected through the skin.

“Medical procedure” or “procedure” includes, but is not limited to, surgical procedures like ablation, diagnostic procedures like biopsies, and therapeutic procedures like drug-delivery.

“Interventional device” includes, but is not limited to, a biopsy needle, a catheter, a guide wire, a laser guide, a device guide, an ablative device, and so on.

“Computer-readable medium”, as used herein, refers to a medium that participates in directly or indirectly providing signals, instructions and/or data. A computer-readable medium may take forms, including, but not limited to, non-volatile media, volatile media, and transmission media. Common forms of a computer-readable medium include, but are not limited to, a floppy disk, a hard disk, a magnetic tape, a CD-ROM, other optical media, a RAM, a memory chip or card, a carrier wave/pulse, and other media from which a computer, a processor or other electronic device can read. Signals used to propagate instructions or other software over a network, like the Internet, can be considered a “computer-readable medium.”

“Data store”, as used herein, refers to a physical and/or logical entity that can store data. A data store may be, for example, a database, a table, a file, a list, a queue, a heap, a memory, a register, and so on. A data store may reside in one logical and/or physical entity and/or may be distributed between two or more logical and/or physical entities.

“Logic”, as used herein, includes but is not limited to hardware, firmware, software and/or combinations of each to perform a function(s) or an action(s), and/or to cause a function or action from another logic, method, and/or system. A logic may take forms including a software controlled microprocessor, a discrete logic like an application specific integrated circuit (ASIC), a programmed logic device, a memory device containing instructions, and so on. A logic may include one or more gates, combinations of gates, or other circuit components. Where multiple logical logics are described, it may be possible to incorporate the multiple logical logics into one physical logic. Similarly, where a single logical logic is described, it may be possible to distribute that single logical logic between multiple physical logics.

An “operable connection”, or a connection by which entities are “operably connected”, is one in which signals, physical communications, and/or logical communications may be sent and/or received. Typically, an operable connection includes a physical interface, an electrical interface, and/or a data interface, but it is to be noted that an operable connection may include differing combinations of these or other types of connections sufficient to allow operable control. For example, two entities can be operably connected by being able to communicate signals to each other directly or through one or more intermediate entities like a processor, operating system, a logic, software, or other entity. Logical and/or physical communication channels can be used to create an operable connection.

“Software”, as used herein, includes but is not limited to, one or more computer or processor instructions that can be read, interpreted, compiled, and/or executed and that cause a computer, processor, or other electronic device to perform functions, actions and/or behave in a desired manner. The instructions may be embodied in various forms like routines, algorithms, modules, methods, threads, and/or programs including separate applications or code from dynamically and/or statically linked libraries. Software may also be implemented in a variety of executable and/or loadable forms including, but not limited to, a stand-alone program, a function call (local and/or remote), a servelet, an applet, instructions stored in a memory, part of an operating system or other types of executable instructions. It will be appreciated that the form of software may depend, for example, on requirements of a desired application, the environment in which it runs, and/or the desires of a designer/programmer or the like. It will also be appreciated that computer-readable and/or executable instructions can be located in one logic and/or distributed between two or more communicating, co-operating, and/or parallel processing logics and thus can be loaded and/or executed in serial, parallel, massively parallel and other manners.

Suitable software for implementing the various components of the example systems and methods described herein may be produced using programming languages and tools like Java, C++, assembly, firmware, microcode, and/or other languages and tools. Software, whether an entire system or a component of a system, may be embodied as an article of manufacture and maintained or provided as part of a computer-readable medium as defined previously. Another form of the software may include signals that transmit program code of the software to a recipient over a network or other communication medium. Thus, in one example, a computer-readable medium has a form of signals that represent the software/firmware as it is downloaded to a user. In another example, the computer-readable medium has a form of the software/firmware as it is maintained on the server.

“User”, as used herein, includes but is not limited to one or more persons, software, computers or other devices, or combinations of these.

Some portions of the detailed descriptions that follow are presented in terms of algorithms and symbolic representations of operations on data bits within a memory. These algorithmic descriptions and representations are the means used by those skilled in the art to convey the substance of their work to others. An algorithm is here, and generally, conceived to be a sequence of operations that produce a result. The operations may include physical manipulations of physical quantities. Usually, though not necessarily, the physical quantities take the form of electrical or magnetic signals capable of being stored, transferred, combined, compared, and otherwise manipulated in a logic and the like.

It has proven convenient at times, principally for reasons of common usage, to refer to these signals as bits, values, elements, symbols, characters, terms, numbers, or the like. It should be borne in mind, however, that these and similar terms are to be associated with the appropriate physical quantities and are merely convenient labels applied to these quantities. Unless specifically stated otherwise, it is appreciated that throughout the description, terms like processing, computing, calculating, determining, displaying, or the like, refer to actions and processes of a computer system, logic, processor, or similar electronic device that manipulates and transforms data represented as physical (electronic) quantities.

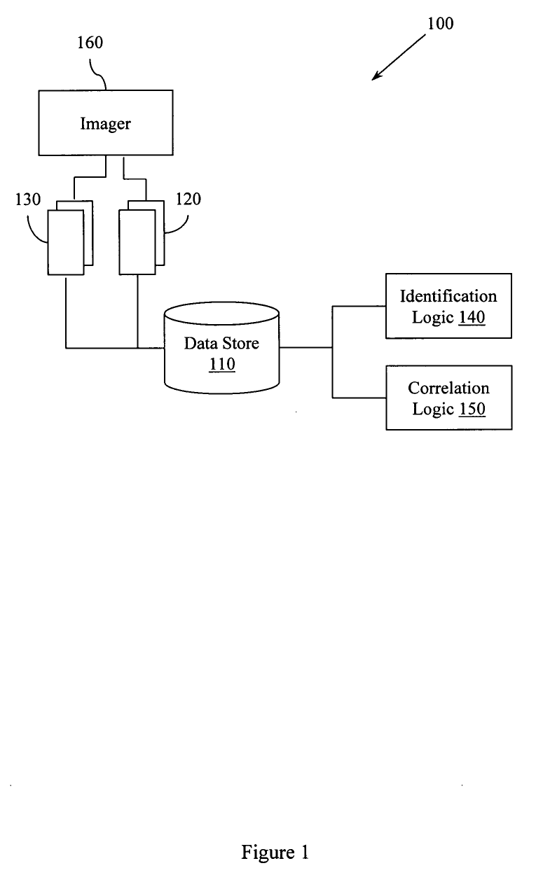

FIG. 1 illustrates an example system 100 that is configured to facilitate intra-procedurally determining the position of an internal anatomical target location using an externally measurable parameter. As described above, in one example, the determining includes identifying and characterizing relationships between the location of a piece of internal anatomy like a suspected tumor and the location of external markers. The external markers may be, for example, coupled MR/optical reference markers that facilitate acquiring position information during both pre-procedural MR imaging and intra-procedural optical imaging. Thus, in one example, an MR/optical reference marker may include an active, capacitively coupled MR marker and a near infrared (IR) optical marker arranged together so that a rigid coordinate transformation exists between the MR marker and the optical marker. The rigid coordinate transformation facilitates a pre-procedural calibration step that establishes a transformation between the MR markers and the optical markers. In another example, an MR/optical reference marker may include an active, inductively coupled MR marker and/or a tuned coil MR marker and a visual light spectrum optical marker arranged together so that a rigid coordinate transformation exists between the MR marker and the optical marker. A tuned coil MR marker refers to the resonant frequency of the MR marker matching the resonant frequency of the MR scanner. In one example, the MR marker and the optical marker may be fabricated into a single assembly where the MR marker and the optical marker maintain fixed positions and orientations with respect to each other.

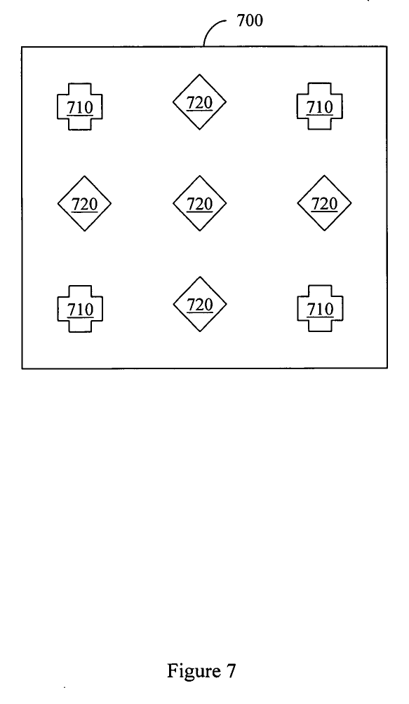

While active capacitively coupled MR markers, active inductively coupled MR markers, tuned coil MR markers, near IR optical markers, and visible light spectrum optical markers are described, it is to be appreciated that other MR markers (e.g., chemical shift) and other optical markers may be employed. One example coupled MR/optical marker is illustrated in FIG. 7. While coupled MR/optical markers are illustrated, it is to be appreciated that other information may be gathered pre-procedurally and/or intra-procedurally from other devices to facilitate accurately predicting an internal target anatomy location, motion, position, and so on. In one example, a device like a chest volume measurement apparatus may be used in addition to and/or in place of coupled MR/optical markers. The apparatus may facilitate acquiring a one dimensional data set related to the amount of air in the lungs and thus to a related chest volume and then, in turn, to an internal anatomical target position. One example apparatus includes a hollow, air filled belt that wraps around the chest of a subject. As the subject inhales and exhales the belt expands and contracts and resulting changes in the air pressure in the belt can be detected and measured. While the data provided by a device like a chest volume measurement apparatus is one dimensional, it may still provide additional data that facilitates improving correlations between internal anatomical target positions and external intra-procedurally measurable parameters.

System 100 may be configured to compensate for the motion of internal anatomical targets if there are observable external parameters (e.g., marker locations) that vary within a finite range like a one, two, or three dimensional space, and if there is a one-to-one (e.g., monotonic) relationship between the observable external parameters and the position and/or motion of the internal anatomical target. The motion may be due to repetitive actions like respiration and/or non-repetitive actions.

System 100 may include a data store 110 that is configured to receive a set of pre-procedural MR images 120 of a subject (not illustrated) from an imager 160. Data store 110 may also be configured to receive other pre-procedural data 130 like chest volume data, ECG data, EMG data, and so on. Imager 160 may be, for example, an MRI apparatus. In one example, before the pre-procedural images are acquired, the subject will have had a set of coupled MR/optical markers positioned on, in, and/or about the subject. For example, a set of markers may be affixed to the chest of the subject chest and stomach area and to a table or platform upon which the subject is located. Thus, when the pre-procedural MR images 120 are acquired, they will include a signal from the MR marker portion of the coupled MR/optical markers. The pre-procedural images are taken to facilitate locating a subcutaneous region of interest (e.g., suspected tumor), tracking its position during a motion (e.g., during respiration), tracking the motion of the coupled MR/optical markers during the same time, and correlating the motion of the internal region to the motion of the external markers. While external markers are described, it is to be appreciated that other externally measurable parameters may be acquired and used in the correlating.

System 100 may include an identification logic 140 that is configured to identify the subcutaneous region of interest in the subject in the set of pre-procedural MR images 120. The region may be three dimensional and thus may move in several directions during, for example respiration. By way of illustration, the region may move up and down in a z axis, left and right in an x axis, and forward and backwards along a y axis. Additionally, the region may deform during, for example, respiration. By way of illustration, as the subject inhales the region may expand while as the subject exhales the region may contract. Thus, identifying the region of interest in the subject in the set of pre-procedural MR images may include determining attributes like a location in an (x,y,z) coordinate system, a size in an (x,y,z) coordinate system, a shape, and so on.

System 100 may also include a correlation logic 150 that is configured to correlate the position of the region of interest as illustrated in the set of pre-procedural MR images 120 with the externally measurable parameters. For example, correlation logic 150 may correlate the position of the region of interest as illustrated in the set of pre-procedural MR images 120 with the position of the set of coupled MR/optical reference markers as illustrated in the set of pre-procedural MR images 120. Correlating the position of the region of interest with the location(s) of members of the set of coupled MR/optical reference markers may include analyzing multivariate data and thus, in one example, principal component analysis (PCA) may be employed to examine the data associated with the pre-procedural images.

PCA may facilitate identifying and characterizing the primary modes of motion in common between the internal anatomical target and the external marker set. More generally, PCA may facilitate identifying and characterizing relationships between the internal anatomical target position and the externally observable and measurable parameters. Understanding the primary modes of motion or other correlations as characterized by PCA (or other numerical analysis techniques) facilitates selecting an appropriate pre-procedural MR image to display during a procedure. For example, as a patient breathes during a procedure, a correlation between an externally measurable parameter (e.g., chest volume, optical marker) may facilitate selecting a pre-procedural MR image to display so that the position of the internal anatomical target, as represented in the selected image, is within a desired distance (e.g., 1.5 mm) of the actual position of the internal anatomical target.

It is to be noted that example systems and methods do not require a patient to breathe in a certain way (e.g., shallowly) or to hold their breath like some conventional systems. In different examples, a patient may be instructed to breath in multiple modes (e.g., normally, deeply, shallowly, rapidly) during pre-procedural imaging to facilitate accommodating these different modes during intra-procedural processing. Thus, it is to be appreciated that example systems and methods may not require restricting the way in which a patient may breath (e.g., breath rate, breathing consistency, depth of inhalation/exhalation).

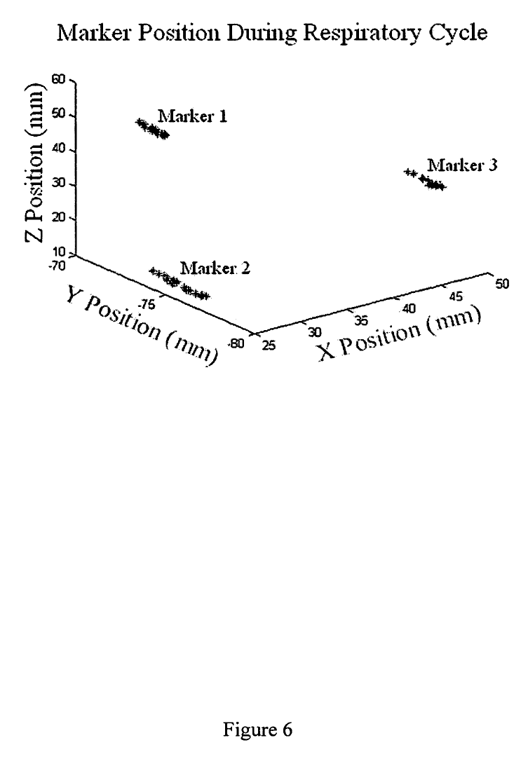

System 100 may be configured to acquire both pre-procedural MR images 120 and other pre-procedural data 130. For example, system 100 may be operably connected to an ECG, an EMG, a chest volume analyzer, and so on. Thus, system 100 may include a control logic (not illustrated) that is configured to control imager 160 (e.g., an MRI apparatus) to acquire MR images substantially simultaneously with other pre-procedural data. In one example, a control circuit that regulates radio frequency (RF) and/or magnetic pulses from imager 160 may also control the read circuitry on another apparatus (e.g., chest volume analyzer). Therefore, as a patient experiences a motion due to, for example, cardiac activity, both MR images and other data (e.g., ECG data) can be acquired. The MR images may facilitate tracking the motion of the MR/optical markers during the motion. That is, it may be possible for the control logic to enable imaging data acquisition to ensure that pre-procedure images are acquired over a wide range of breathing and/or motion conditions, or that imaging continues until images associated with a sufficiently wide range of motions and/or configurations are acquired. For example, in FIG. 6, a plot of the motion of three markers during a respiratory cycle is provided. While three markers are illustrated, it is to be appreciated that a greater number of markers may be employed. Furthermore, while respiration is described, other motion like that described above may be analyzed.

Different numbers and series of MR images 120 may be acquired for different procedures. In one example, the set of pre-procedural MR images 120 may include at least sixteen images taken at substantially evenly spaced time intervals throughout a respiratory cycle. Similarly, the set of pre-procedural data 130 may also include readings taken at times corresponding to the times at which the pre-procedural MR images 120 are acquired. While sixteen MR images are described, it is to be appreciated that a greater and/or lesser number of images may be acquired. In one example, the MR images 120 and the pre-procedural data 130 may be acquired at almost the exact same time if an external device (e.g., ECG) and the MR imager are operably connected. In another example, the MR images 120 and the other pre-procedural data 130 may be acquired in an alternating sequence with a period of time elapsing between each acquisition. Thus, in this context, “times corresponding to” and “substantially simultaneously” refer to acquiring two sets of data (e.g., MR image, chest volume reading) at points in time sufficiently close together so that a position and/or movement correlation is possible. In one example, this means the acquisitions are taken within a time period less than one sixteenth of the time it takes to complete the motion. In another example, this means the acquisitions are taken within a time period less than the amount of time it takes for either the region of interest or an external marker to travel a distance greater than the accuracy (e.g., 2 mm) of the system. It is to be appreciated that motion may not be periodic. Thus, data may be collected over a sufficient time frame to ensure coverage of a wide range of conditions associated with non-periodic motion.

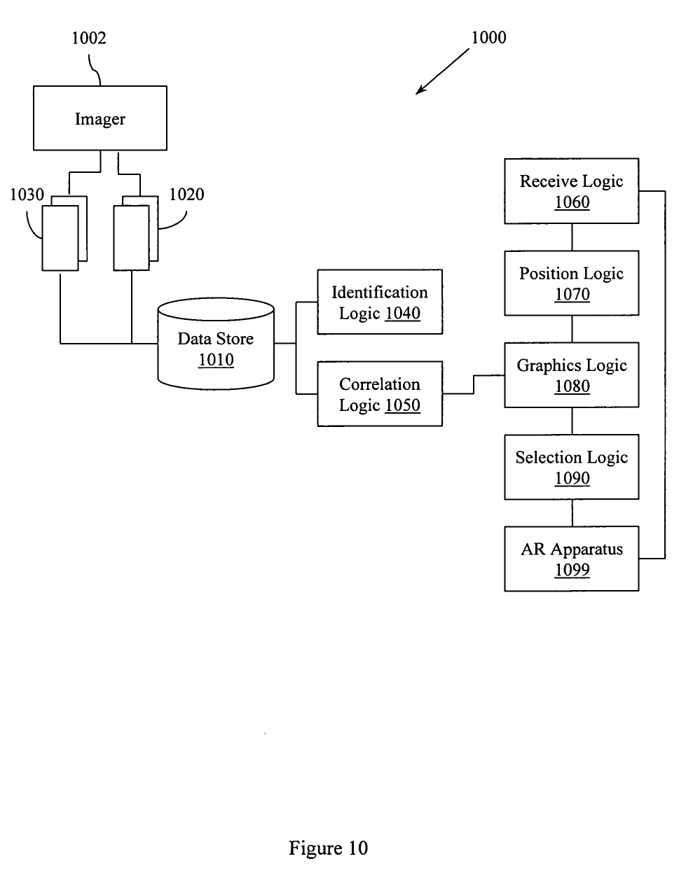

With the correlation between internal anatomical position and externally measurable parameters complete, information for guiding a percutaneous procedure may now be generated for an augmented reality (AR) or other type system like that illustrated in FIG. 10. AR system 1000 includes a data store 1010 configured like data store 110. Similarly, system 1000 includes an imager 1002 like imager 160, an identification logic 1040 like identification logic 140 and a correlation logic 1050 like correlation logic 150.

Additionally, AR system 1000 includes a receive logic 1060 operably connected to an AR apparatus 1099. Receive logic 1060 may be configured to receive, for example, an intra-procedural optical image that includes information concerning both the set of coupled MR/optical reference markers and a set of visual reference markers rigidly and fixedly coupled to an interventional device (not illustrated). Once again, while coupled MR/optical markers are described, it is to be appreciated that other intra-procedural data like ECG data, EMG data, and so on, may be acquired and employed to select pre-procedural MR images to display. In one example, an intra-procedural optical image will include information from the coupled MR/optical reference markers associated with the subject and also from the interventional device. The intra-procedural optical image can provide data for the relations identified by correlation logic 1050. Thus, the intra-procedural optical image can facilitate inferring the location of the internal region of interest from the position of the coupled MR/optical reference markers. Furthermore, the intra-procedural optical image can also facilitate inferring the position of the interventional device relative to that internal region of interest.

To facilitate locating, positioning, and/or tracking the interventional device, AR system 1000 may include a position logic 1070 that is configured to establish a position of the interventional device in a coordinate framework that includes the set of coupled MR/optical reference markers and the subject. In one example, the coordinate framework may be, for example, a three dimensional framework (x,y,z) with its origin at a fixed point like an MR and optically visible point on a scanner bed. In another example, the coordinate framework may be a four dimensional framework (x,y,z,t) with its origin centered in the center of mass of the region of interest at time t₀. While two coordinate frameworks are described, it is to be appreciated that other frameworks may be employed. In one example, imager 1002 and the AR apparatus 1099 facilitate locating the region of interest, the interventional device, and/or an external marker to within 2 mm.

AR system 1000 may also include a graphics logic 1080 that is configured to produce a computer generated image of the interventional device during the percutaneous procedure. Since the interventional device is likely to enter the subject during the procedure, the computer generated image may include a representation of the portion of the interventional device located inside the subject.

During the procedure, it may be appropriate to display to the interventionalist (e.g., surgeon, physician, technician, assistant) different information at different times. For example, while the device is moving the interventionalist may want to see anatomy in the path of the device and whether the device is getting closer to or farther away from the region of interest, a desired device track, and so on. Similarly, while the device is not moving the interventionalist may want to see a survey of the internal anatomy around the tool for a period of time and also the actual skin surface of the patient to check, for example, for excessive bleeding. Thus, system 1000 may include a selection logic 1090 that is configured to select a pre-procedural MR image to provide to AR apparatus 1099 based, at least in part, on the intra-procedural optical image. While an intra-procedural optical image is described, it is to be appreciated that other intra-procedural data may be acquired from other systems like an x-ray system, a fluoroscopy system, an ultrasound system, an endoscopic system, and so on. Selection logic 1090 may also be configured to selectively combine the computer generated image of the interventional device provided by graphics logic 1080 with the pre-procedural MR image to make a sophisticated, information rich presentation for the interventionalist. In one example, the graphics may be overlaid on an optical image acquired by the AR system 1000 while in another example the graphics may be overlaid on x-ray images, fluoroscopic images, and so on.

The presentation may be made, for example, by AR apparatus 1099. AR apparatus 1099 may include, for example, a stereoscopic display with video-see-through capability. Thus, the interventionalist may see the subject using the see-through capability but may also be presented with additional information like computer graphics associated with the underlying anatomy, the interventional device, and so on. In one example, the stereoscopic display may be head-mountable.

AR apparatus 1099 may also include a video camera based stereoscopic vision system configured to acquire an intra-procedural visual image of the subject. This may be thought of as being “artificial eyes” for the interventionalist. In one example, the video camera may facilitate magnifying the object being observed. Thus, in some examples, a stereoscopic display may selectively display a magnified view rather than a real-world view.

AR apparatus 1099 may also include a camera (e.g., a tracking camera) that is configured to acquire the intra-procedural optical image that includes information concerning both the set of coupled MR/optical reference markers and the set of visual reference markers associated with the interventional device. In one example, the tracking camera may operate in the visible light spectrum while in another example the camera may operate in other ranges like the near-IR range. In one example, when the tracking camera operates in the visible light spectrum it may be combined with the stereoscopic vision system.

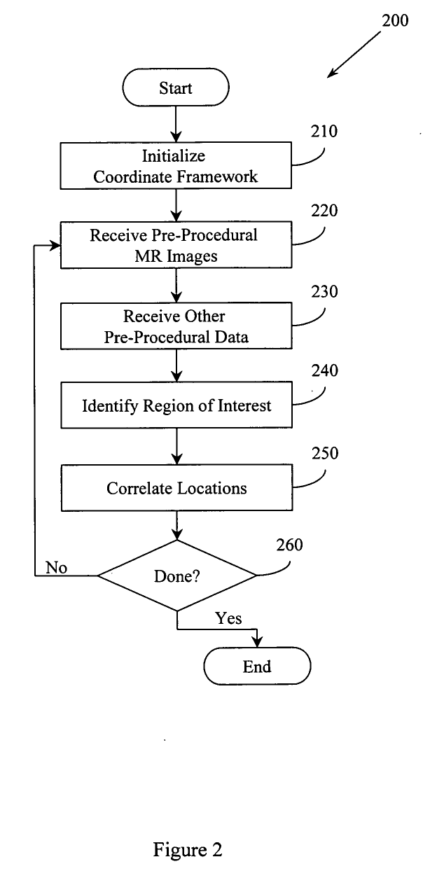

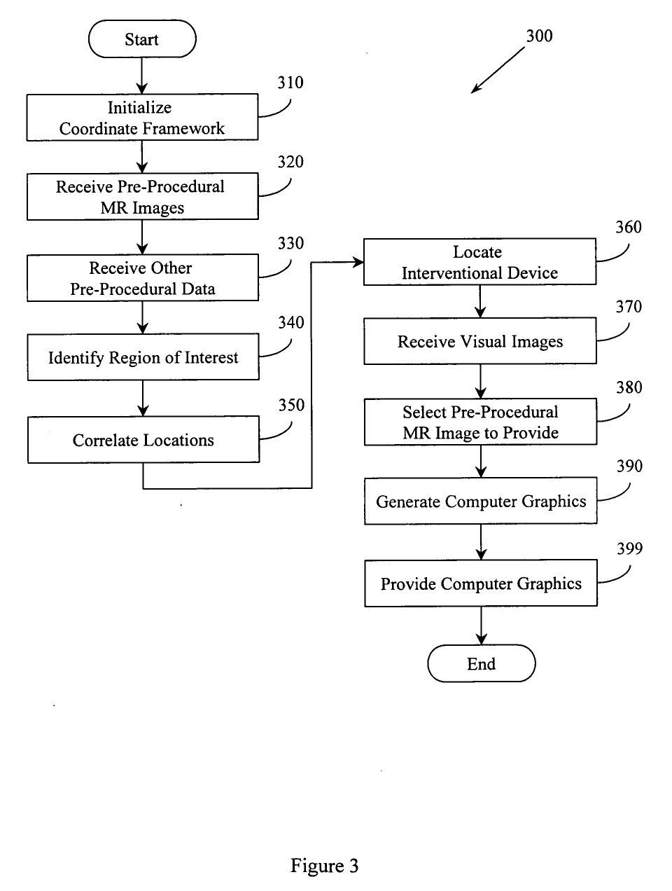

Example methods may be better appreciated with reference to the flow diagrams of FIGS. 2 and 3. While for purposes of simplicity of explanation, the illustrated methodologies are shown and described as a series of blocks, it is to be appreciated that the methodologies are not limited by the order of the blocks, as some blocks can occur in different orders and/or concurrently with other blocks from that shown and described. Moreover, less than all the illustrated blocks may be required to implement an example methodology. Furthermore, additional and/or alternative methodologies can employ additional, not illustrated blocks.

In the flow diagrams, blocks denote “processing blocks” that may be implemented with logic. The processing blocks may represent a method step and/or an apparatus element for performing the method step. A flow diagram does not depict syntax for any particular programming language, methodology, or style (e.g., procedural, object-oriented). Rather, a flow diagram illustrates functional information one skilled in the art may employ to develop logic to perform the illustrated processing. It will be appreciated that in some examples, program elements like temporary variables, routine loops, and so on, are not shown. It will be further appreciated that electronic and software applications may involve dynamic and flexible processes so that the illustrated blocks can be performed in other sequences that are different from those shown and/or that blocks may be combined or separated into multiple components. It will be appreciated that the processes may be implemented using various programming approaches like machine language, procedural, object oriented and/or artificial intelligence techniques.

FIG. 2 illustrates an example computer-executable method 200 associated with providing real time computer graphics for guiding a percutaneous procedure without employing real time intra-procedural (e.g., MR) imaging. Method 200 may include, at 210, initializing a coordinate framework like an (x,y,z,t) framework. The (x,y,z,t) framework may facilitate describing the relative locations (x,y,z) of items at different times (t). For example, the framework may facilitate identifying the location of a subject, a region of interest inside the subject, an interventional device, members of a set of coupled MR/optical markers associated with the subject, and so on, at different times. The times may include, for example, different points in a repetitive motion cycle like respiration, different points during non-periodic motion, and so on.

Method 200 may also include, at 220, receiving pre-procedural MR images that include a first data about the coupled MR/optical markers. This data may be, for example, simply the recorded image of the MR marker from which its (x,y,z) position can be determined relative to other markers, an internal region of interest, a fixed point, and so on. Unlike conventional systems, the subject is not expected to breathe in any restricted way during both the pre-procedural data collection and later, during the procedure.

Method 200 may also include, at 230, receiving other pre-procedural data. This data may be, for example, from a chest volume measuring apparatus, an ECG, an EMG, and so on. In some examples, no other pre-procedural data may be acquired and 230 may be omitted.

Method 200 may also include, at 240, identifying the region of interest inside the subject as illustrated in the pre-procedural MR images. The identifying may include, for example, receiving an input from a user (e.g., oncologist) who outlines the region in various images. The identifying may also include, for example, receiving an input from an artificial intelligence system configured to identify abnormal or suspicious areas. While manual input and artificial intelligence input are described, it is to be appreciated that the region of interest may be identified by other techniques.

Method 200 may also include, at 250, correlating the location of the region of interest with the location of the coupled MR/optical markers at different points in time. These different points in time may correspond, for example, to different locations of the region of interest as it moves. The correlating will be achieved by analyzing the first data. In some examples, when other pre-procedural data is available, it may also be analyzed in the correlating step. The first data (and the other pre-procedural data) may be sets of (x,y,z,t) multivariate data that can be processed using principal component analysis (PCA) to identify and characterize relations between the data. As described above, PCA (and other techniques) facilitate identifying and characterizing, for example, primary modes of motion in common between the internal anatomical target and the external marker set.

While FIG. 2 illustrates various actions occurring in serial, it is to be appreciated that various actions illustrated in FIG. 2 could occur substantially in parallel. By way of illustration, a first process could initialize the coordinate framework while a second process could receive the pre-procedural MR images, a third process could be tasked with identifying a region of interest in the MR images and a fourth process could perform the correlations. While four processes are described, it is to be appreciated that a greater and/or lesser number of processes could be employed and that lightweight processes, regular processes, threads, and other approaches could be employed. It is to be appreciated that other example methods may, in some cases, also include actions that occur substantially in parallel.

In one example, methodologies are implemented as processor executable instructions and/or operations provided on a computer-readable medium. Thus, in one example, a computer-readable medium may store processor executable instructions operable to perform a method for providing real time computer graphics for guiding a percutaneous procedure without employing real time intra-procedural (e.g., MR) imaging. The method may include, for example, initializing a coordinate framework for describing the relative locations of a subject, a region of interest inside the subject, an interventional device, members of a set of coupled MR/optical markers associated with the subject and so on. The method may also include, for example, receiving pre-procedural MR images that include a first data about the coupled MR/optical markers. The first data may facilitate correlating the position and/or movement of an internal region of interest and the external markers. Thus, the method may include identifying the region of interest inside the subject as illustrated in the pre-procedural MR images and correlating the location of the region of interest with the location of the coupled MR/optical markers at various points in time. In one example, the method may also include locating the interventional device in the coordinate framework, receiving visual images of the subject, the coupled MR/optical markers, and the interventional device during the procedure. The method may then include selecting a pre-procedural MR image to provide to an augmented reality apparatus. The method may also include generating computer graphics concerning the interventional device, the region of interest, and so on, and providing the computer graphics to the augmented reality apparatus. While this method is described being provided on a computer-readable medium, it is to be appreciated that other example methods described herein may also be provided on a computer-readable medium.

FIG. 3 illustrates an example computer-executable method 300 associated with providing real time computer graphics for guiding a percutaneous procedure without employing real time intra-procedural (e.g., MR) imaging. Method 300 includes actions 310 through 350 that are similar to actions 210 through 250. Method 300 may also include, at 360, locating an interventional device like a biopsy needle in the coordinate framework. As described above, to facilitate locating and tracking the interventional device it may have optical reference markers attached to it. These optical markers help identifying the location, orientation, and so on of the device during a procedure. But before the device can be tracked during the procedure it first needs to be placed in the coordinate framework and the tracking system calibrated.

Method 300 may also include, at 370, receiving visual images during the procedure. The visual images may include, for example, the subject, the coupled MR/optical markers, the interventional device, reference markers on the device, and so on. Since a transformation was determined between the optical portion of the coupled MR/optical markers and the MR portion of the coupled MR/optical markers, information related to marker position in the pre-procedural images and the intra-procedural images can be used to determine information to present. While 370 describes receiving visual images, it is to be appreciated that other intra-procedural imaging like x-ray, fluoroscopy, and so on may be employed.

Method 300 may also include, at 380, selecting a pre-procedural MR image to provide to an augmented reality apparatus and, at 390, generating computer graphics concerning items like the interventional device, the region of interest, and so on. Selecting the pre-procedural MR image based on correlations between marker positions and intra-procedural images facilitates identifying relevant information for guiding the procedure. For example, since the region of interest may move during the procedure, and since its position may be correlated with external marker position, the position of the region of interest at different points in time and relations to the interventional device at those points in time can be determined from the pre-procedural correlations and data associated with the intra-procedural images. Once again, while intra-procedural images are described, the intra-procedural data may be acquired from other apparatus like an ECG, an EMG, a chest volume measuring apparatus and so on. In these cases, the position of the region of interest may be correlated with non visual data and thus the MR image to display may be selected based on this non visual data.

Method 300 may also include, at 399, providing the computer graphics to an AR apparatus. The computer graphics may include, for example, a rendering of an MRI slice at an interesting level like the device tip level, a graphical target like a homing signal, an actual device track, a desired device track, a projected device track, and so on. In one example, the computer graphics may include overlays, superimpositions, mergings, and so on that include a live stereoscopic video view of real scene and the generated computer graphics.

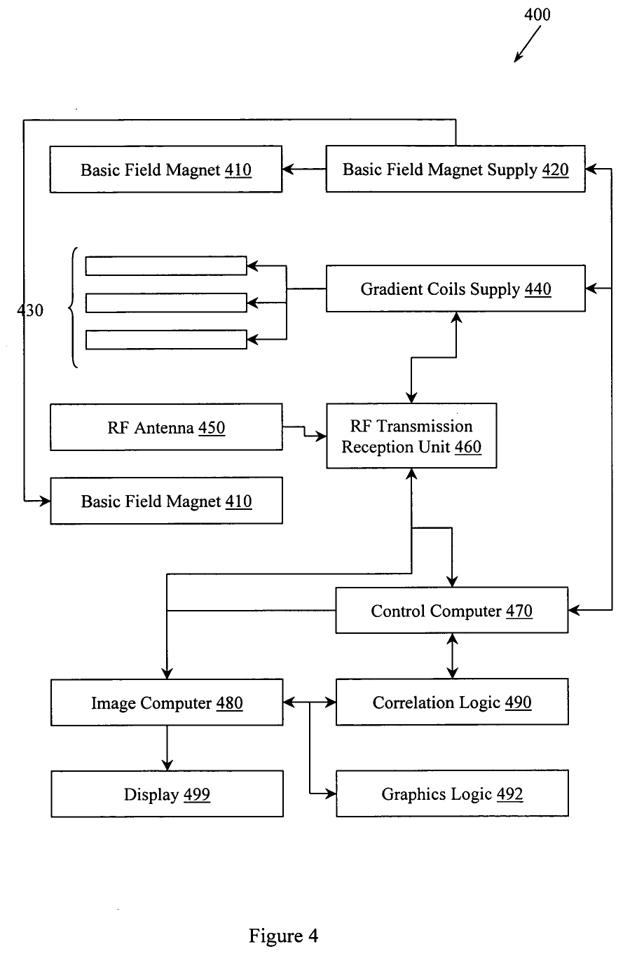

FIG. 4 illustrates an example MRI apparatus 400 configured to facilitate intra-procedurally determining the position of an internal anatomical target location using an externally measurable parameter. Apparatus 400 may be one of many different types of MRI apparatus, for example, a Siemens 1.5T Sonata imager. Apparatus 400 includes a basic field magnet(s) 410 and a basic field magnet supply 420. Ideally, the basic field magnets 410 would produce a uniform B₀ field. However, in practice, the B₀ field may not be uniform, and may vary over an object being imaged by the MRI apparatus 400. MRI apparatus 400 may include gradient coils 430 configured to emit gradient magnetic fields like G_{S}, G_{P} and G_{R}. The gradient coils 430 may be controlled, at least in part, by a gradient coils supply 440.

MRI apparatus 400 may also include an RF antenna 450 that is configured to generate RF pulses and to receive resulting magnetic resonance signals from an object to which the RF pulses are directed. In one example, separate RF transmission and reception coils can be employed. The RF antenna 450 may be controlled, at least in part, by an RF transmission-reception unit 460. The gradient coils supply 440 and the RF transmission-reception unit 460 may be controlled, at least in part, by a control computer 470. In one example, the control computer 470 may be programmed to perform methods like those described herein.

The MR signals received from the RF antenna 450 can be employed to generate an image, and thus may be subject to a transformation process like a two dimensional FFT that generates pixilated image data. The transformation can be performed by an image computer 480 or other similar processing device. In one example, image computer 480 may be programmed to perform methods like those described herein. The image data may then be shown on a display 499.

While FIG. 4 illustrates an example MRI apparatus 400 that includes various components connected in various ways, it is to be appreciated that other MRI apparatus may include other components connected in other ways. In one example, to implement the example systems and methods described herein, MRI apparatus 400 may be configured with a correlation logic 490. In different examples, correlation logic 490 may be permanently and/or removably attached to an MRI apparatus. While correlation logic 490 is illustrated as a single logic connected to control computer 470 and image computer 480, it is to be appreciated that correlation logic 490 may be distributed between and/or operably connected to other elements of apparatus 400. Correlation logic 490 may be configured to receive pre-procedural MR images of a subject and intra-procedural data (e.g., marker position data). Correlation logic 490 may also be configured to correlate the position and/or movements of a region inside the subject with the position and/or movements of markers located, for example, on the subject. MRI apparatus 400 may also include a graphics logic 492 that is configured to receive intra-procedural visual images of the subject and an interventional device and then to produce a computer generated image of the interventional device, the subject, and/or the region inside the subject in which the intervention is to occur.

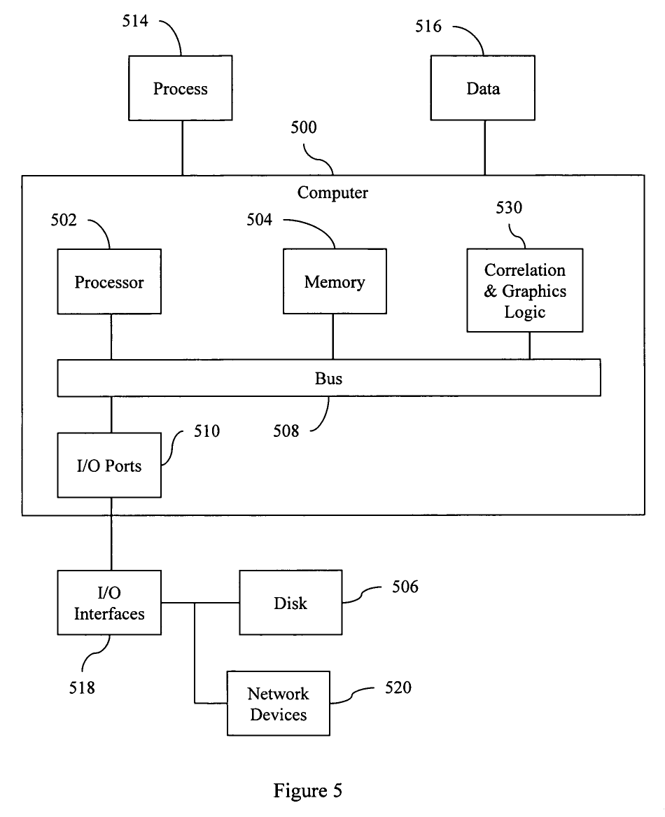

FIG. 5 illustrates an example computer 500 in which example methods illustrated herein can operate and in which example motion correlating logics may be implemented. In different examples computer 500 may be part of an MRI apparatus or may be operably connectable to an MRI apparatus.

Computer 500 includes a processor 502, a memory 504, and input/output ports 510 operably connected by a bus 508. In one example, computer 500 may include a correlation and graphics logic 530 that is configured to facilitate actions like those associated with correlation logic 490 and graphics logic 492. Thus, correlation and graphics logic 530, whether implemented in computer 500 as hardware, firmware, software, and/or a combination thereof may provide means for pre-procedurally correlating the location of an item of internal anatomy as revealed by MR imaging with the location of an external marker as revealed by optical imaging and means for guiding a percutaneous procedure outside an MR imager without acquiring real time MR images during the procedure based, at least in part, on the correlating. In different examples, correlation and graphics logic 530 may be permanently and/or removably attached to computer 500.

Processor 502 can be a variety of various processors including dual microprocessor and other multi-processor architectures. Memory 504 can include volatile memory and/or non-volatile memory. A disk 506 may be operably connected to computer 500 via, for example, an input/output interface (e.g., card, device) 518 and an input/output port 510. Disk 506 can include, but is not limited to, devices like a magnetic disk drive, a tape drive, a Zip drive, a flash memory card, and/or a memory stick. Furthermore, disk 506 may include optical drives like a CD-ROM and/or a digital video ROM drive (DVD ROM). Memory 504 can store processes 514 and/or data 516, for example. Disk 506 and/or memory 504 can store an operating system that controls and allocates resources of computer 500.

Bus 508 can be a single internal bus interconnect architecture and/or other bus or mesh architectures. While a single bus is illustrated, it is to be appreciated that computer 500 may communicate with various devices, logics, and peripherals using other busses that are not illustrated (e.g., PCE, SATA, Infiniband, 1394, USB, Ethernet).

Computer 500 may interact with input/output devices via i/o interfaces 518 and input/output ports 510. Input/output devices can include, but are not limited to, a keyboard, a microphone, a pointing and selection device, cameras, video cards, displays, disk 506, network devices 520, and the like. Input/output ports 510 can include but are not limited to, serial ports, parallel ports, and USB ports.

Computer 500 may operate in a network environment and thus may be connected to network devices 520 via i/o interfaces 518, and/or i/o ports 510. Through the network devices 520, computer 500 may interact with a network. In one example, computer 500 may be connected through a network to the MRI apparatus whose acquisition parameters may be dynamically adapted. Through the network, computer 500 may be logically connected to remote computers. The networks with which computer 500 may interact include, but are not limited to, a local area network (LAN), a wide area network (WAN), and other networks.

FIG. 7 illustrates an example external reference marker 700. The external reference marker 700 includes a set of MR “visible” elements 710 and a set of optically “visible” elements 720. “Visible”, as used herein, refers to the ability of an imaging apparatus (e.g., MRI apparatus, camera) to detect the marker while acquiring an image. In one example, the external reference marker 700 may be fabricated onto a rigid plate made, for example, of plastic. In different examples, the MR visible elements 710 and optical visible elements 720 may be fixedly attached (e.g., glued, fabricated into, fabricated onto) the rigid plate to facilitate establishing a transformation between the two elements. In one example the sets of MR visible elements 710 and optically visible elements 720 may be arranged so that a constant, rigid coordinate transformation can be established between members of the sets. While four MR elements 710 and five optical elements 720 are illustrated, it is to be appreciated that a greater and/or lesser number of elements arranged in different patterns may be employed. It is to be appreciated that the MR elements 710 may take different forms including, for example, inductively coupled elements, capacitively coupled elements, RF tuned elements, chemical shift elements, and so on.

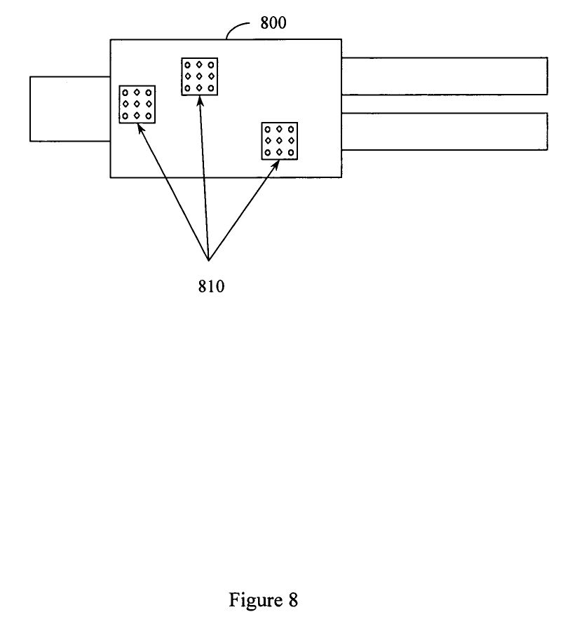

FIG. 8 illustrates a subject 800 with which a set of motion tracking markers 810 has been associated. Associating the tracking markers 810 with the subject may include, for example, placing a marker on a patient, affixing (e.g., gluing, sewing, stapling, screwing) the marker to a patient, and so on. While a human is illustrated as subject 800, it is to be appreciated that some example systems and methods described herein may be employed in other (e.g., veterinary) applications.

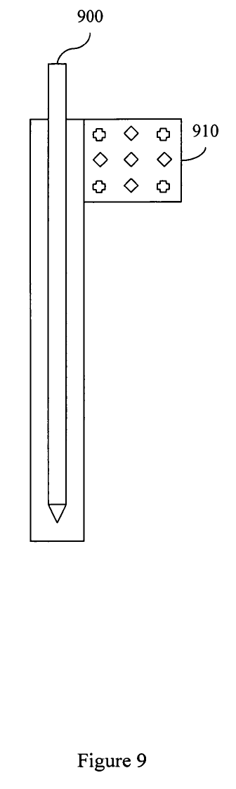

FIG. 9 illustrates an interventional device 900 to which a motion tracking marker 910 has been attached. Attaching marker 910 to device 900 facilitates locating device 900, establishing its initial position in a coordinate framework, and tracking it during a procedure. While a single marker 910 is illustrated, it is to be appreciated that one or more markers 910 may be attached to a device 900. The device 900 may be, for example, a biopsy needle, an arthroscopic device, a micro-scalpel, a guide-wire, and so on.

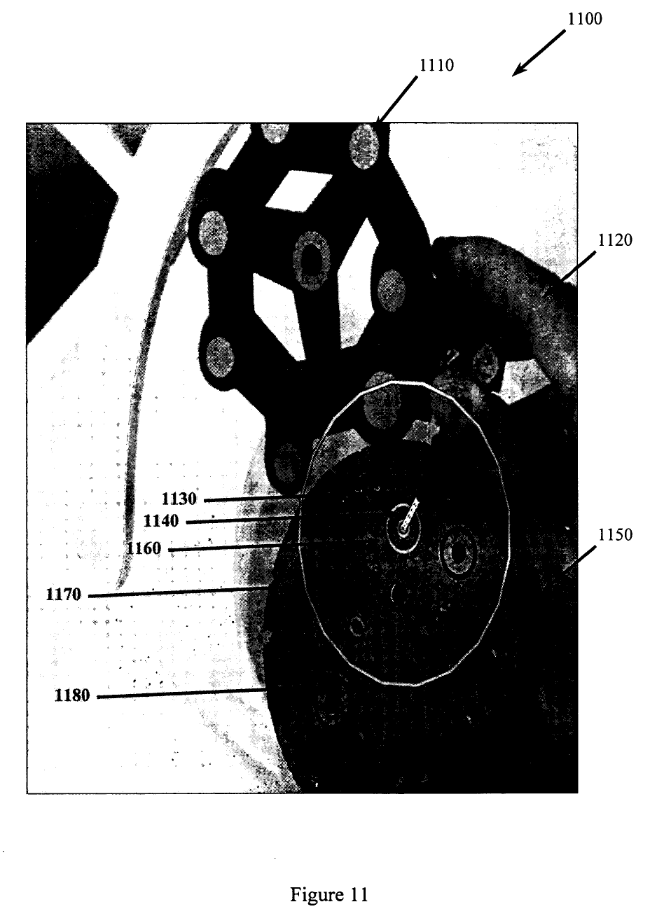

FIG. 11 illustrates an example screenshot 1100 from an example AR system. Screenshot 1100 includes image 1110 of a reference marker, image 1120 of a hand of an interventionalist, and image 1130 of a visible portion of an interventional device, in this case a biopsy needle. These images may be acquired using, for example, a video camera based stereoscopic vision system associated with an augmented reality system. These images may be displayed, for example, on a stereoscopic display with video-see-through capability. Thus, in some examples, the images may simply be what the interventionalist sees through the stereoscopic display.

Screenshot 1100 also includes an MR image 1150. MR image 1150 would have been acquired pre-procedurally. The augmented reality system may have selected MR image 1150 to display based, for example, on the position of interventional device 1130 as determined by the location of reference marker 1110 and the position of other external reference markers that provide information concerning the likely position of an internal region of interest.

Screen shot 1100 also includes an image of a visible portion of interventional device 1130 and a computer generated graphic of a portion 1140 of interventional device 1130 located inside a subject. The computer generated graphic of portion 1140 illustrates where the tip of device 1130 is with respect to anatomy (e.g., suspected tumor) illustrated in MR image 1150. Additionally, computer graphic 1160 illustrates a target region towards which interventional device 1130 should be directed and range feedback graphic 1170 that facilitates understanding how far from target region 1160 the interventional device 1130 is located. Screenshot 1100 also includes a graphic 1180 that indicates that another region illustrated in MR image 1150 has already been processed by interventional device 1130. This may facilitate an interventionalist not acquiring two samples from a single region and so on. While a needle biopsy, MR slice, target graphics, and so on are illustrated, it is to be appreciated that other images, graphics, and so on may be employed.

While example systems, methods, and so on, have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and so on, described herein. Additional advantages and modifications will readily appear to those skilled in the art. Therefore, the invention is not limited to the specific details, the representative apparatus, and illustrative examples shown and described. Thus, this application is intended to embrace alterations, modifications, and variations that fall within the scope of the appended claims. Furthermore, the preceding description is not meant to limit the scope of the invention. Rather, the scope of the invention is to be determined by the appended claims and their equivalents.

To the extent that the term “includes” or “including” is employed in the detailed description or the claims, it is intended to be inclusive in a manner similar to the term “comprising” as that term is interpreted when employed as a transitional word in a claim. Furthermore, to the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Gamer, A Dictionary of Modern Legal Usage 624 (2d. Ed. 1995).

What is claimed is:

1. A system, comprising:
a data store configured to receive a set of pre-procedural magnetic resonance (MR) images of a subject that includes information concerning a set of coupled MR/optical reference markers associated with the subject;
an identification logic configured to identify a subcutaneous region of interest in the subject in the set of pre-procedural MR images; and
a correlation logic configured to correlate the position of the region of interest as illustrated in the set of pre-procedural MR images with the position of the set of coupled MR/optical reference markers as illustrated in the set of pre-procedural MR images.

2. The system of claim 1, including:
a receive logic configured to receive an intra-procedural optical image that includes information concerning both the set of coupled MR/optical reference markers and a set of visual reference markers rigidly and fixedly coupled to an interventional device;
a position logic configured to establish a position of the interventional device in a coordinate framework that includes the set of coupled MR/optical reference markers and the subject;
a graphics logic configured to produce a computer generated image of the interventional device, including a portion of the interventional device located inside the subject; and
a selection logic configured to select a member of the set of pre-procedural MR images to provide to an augmented reality (AR) apparatus based, at least in part, on the intra-procedural optical image, and to selectively combine the computer generated image of the interventional device with the selected pre-procedural MR image.

3. The system of claim 2, including a control logic configured to control an MRI apparatus to acquire a pre-procedural MR image and to control an external device to acquire a pre-procedural data substantially simultaneously with the acquisition of a corresponding pre-procedural MR image.

4. The system of claim 3, the set of pre-procedural MR images including at least sixteen images taken at substantially evenly spaced time intervals throughout a movement of the region of interest, the movement being one of periodic, and not periodic.

5. The system of claim 2, where the interventional device, the set of coupled MR/optical reference markers, and the region of interest can be located to within 2 mm in the coordinate framework.

6. The system of claim 2, where the set of coupled MR/optical reference markers includes one or more active, capacitively coupled MR markers and one or more near infrared optical markers arranged together so that a rigid coordinate transformation exists between the MR markers and the optical markers.

7. The system of claim 2, where the set of coupled MR/optical reference markers includes one or more active, inductively coupled markers and one or more near infrared optical markers arranged together so that a rigid coordinate transformation exists between the MR markers and the optical markers.

8. The system of claim 2, the set of coupled MR/optical reference markers including a tuned coil MR marker.

9. The system of claim 2, the AR apparatus including a stereoscopic display with video-see-through capability.

10. The system of claim 9, the stereoscopic display being head-mountable.

11. The system of claim 9, the AR apparatus including a video camera based stereoscopic vision system configured to acquire an intra-procedural visual image of the subject.

12. The system of claim 11, the AR apparatus including an optical tracking camera configured to acquire the intra-procedural optical image that includes information concerning both the set of coupled MR/optical reference markers and the set of visual reference markers.

13. The system of claim 12, the optical tracking camera being configured to acquire the intra-procedural optical image using one or more of, an x-ray apparatus, a fluoroscopic apparatus, an endoscopic apparatus, and an ultrasound apparatus.

14. The system of claim 1, where the system is incorporated into an MRI apparatus.

15. An apparatus, comprising:
an MRI apparatus;
a data store configured to receive a set of pre-procedural magnetic resonance (MR) images of a subject that include information concerning a set of coupled MR/optical reference markers associated with the subject;
an identification logic configured to identify a subcutaneous region of interest in the subject in the set of pre-procedural MR images;
a correlation logic configured to correlate the position of the region of interest as illustrated in the set of pre-procedural MR images with the position of the set of coupled MR/optical reference markers as illustrated in the set of pre-procedural MR images;
a receive logic configured to receive an intra-procedural optical image that includes information concerning both the set of coupled MR/optical reference markers and a set of visual reference markers rigidly and fixedly coupled to an interventional device;
a position logic configured to establish a position of the interventional device in a coordinate framework that includes the set of coupled MR/optical reference markers and the subject;
a graphics logic configured to produce a computer generated image of the interventional device, including a portion of the interventional device located inside the subject; and
a selection logic configured to select a member of the set of pre-procedural MR images to provide to an augmented reality (AR) apparatus based, at least in part, on the intra-procedural optical image, and to selectively combine the computer generated image of the interventional device with the selected pre-procedural MR image,
the AR apparatus comprising:
a stereoscopic display with video-see-through capability;
a video camera based stereoscopic vision system configured to acquire an intra-procedural visual image of the subject; and
an optical tracking camera configured to acquire the intra-procedural optical image that includes information concerning both the set of coupled MR/optical reference markers and the set of visual reference markers.

16. A computer-implemented method for providing real time computer graphics for guiding a percutaneous procedure without employing real time intra-procedural imaging, comprising:
initializing a coordinate framework for describing the relative locations of a subject, a region of interest inside the subject, an interventional device, and a set of coupled MR/optical markers associated with the subject;
receiving pre-procedural MR images that include a first data concerning the set of coupled MR/optical markers;
identifying the region of interest inside the subject as illustrated in the pre-procedural MR images; and
correlating the location of the region of interest with the location of members of the set of coupled MR/optical markers at two or more points in time corresponding to two or more different locations of the region of interest based, at least in part, on the first data.

17. The method of claim 16, including:
locating the interventional device in the coordinate framework;
receiving visual images of the subject, the set of coupled MR/optical markers, and the interventional device during the procedure;
selecting a pre-procedural MR image to provide to an augmented reality apparatus;
generating computer graphics concerning the interventional device and the region of interest; and
providing the computer graphics to the augmented reality apparatus.

18. The method of claim 16, where initializing the coordinate framework includes establishing a relation between one or more moveable elements and one or more fixed points.

19. The method of claim 16, where the pre-procedural MR images cover one or more cycles of a repetitive motion of the subject.

20. The method of claim 19, the cycles being associated with one or more of, respiration, and cardiac activity.

21. The method of claim 18, where the pre-procedural MR images cover a span of time in which a non-periodic motion occurs.

22. The method of claim 16, including receiving a second pre-procedural data from one or more of, an electrocardiogram, an electromyogram, and a chest volume measuring apparatus.

23. The method of claim 16, the first data comprising multivariate data and where correlating the location of the region of interest with the location of members of the set of coupled MR/optical markers is performed using principal component analysis (PCA) on the pre-procedural MR images.

24. A computer-readable medium storing computer-executable instructions operable to perform a computer-implemented method for providing real time computer graphics for guiding a percutaneous procedure without employing real time intra-procedural imaging, comprising:
initializing a coordinate framework for describing the relative locations of a subject, a region of interest inside the subject, an interventional device, and a set of coupled MR/optical markers associated with the subject;
receiving pre-procedural MR images that include a first data concerning the set of coupled MR/optical markers;
identifying the region of interest inside the subject as illustrated in the pre-procedural MR images;
correlating the location of the region of interest with the location of members of the set of coupled MR/optical markers at two or more points in time corresponding to two or more different locations of the region of interest based, at least in part, on the first data;
locating the interventional device in the coordinate framework;
receiving visual images of the subject, the set of coupled MR/optical markers, and the interventional device during the procedure;
selecting a pre-procedural MR image to provide to an augmented reality apparatus;
generating computer graphics concerning the interventional device and the region of interest; and
providing the computer graphics to the augmented reality apparatus.

25. An apparatus, comprising:
means for pre-procedurally correlating the location of an item of internal anatomy as revealed by magnetic resonance imaging with an externally measurable parameter; and
means for guiding a percutaneous procedure outside a magnetic resonance imager without acquiring real time magnetic resonance images during the procedure based, at least in part, on the correlating.

26. A system, comprising:
a data store configured to receive a set of pre-procedural magnetic resonance (MR) images of a subject that includes information concerning a set of MR reference markers affixed to the subject;
an identification logic configured to identify a subcutaneous region of interest in the subject in the set of pre-procedural MR images; and
a correlation logic configured to correlate the position of the region of interest as illustrated in the set of pre-procedural MR images with an externally intra-procedurally measurable parameter.

27. The system of claim 26, including:
a receive logic configured to receive data concerning the externally intra-procedurally measurable parameter;
a position logic configured to establish a position of the interventional device in a coordinate framework that includes the subject;
a graphics logic configured to produce a computer generated image of the interventional device, including a portion of the interventional device located inside the subject; and
a selection logic configured to select a member of the set of pre-procedural MR images to provide to an augmented reality (AR) apparatus based, at least in part, on the data concerning the externally intra-procedurally measurable parameter, and to selectively combine the computer generated image of the interventional device with the selected pre-procedural MR image.
